(19) United States
(12) Patent Application Publication
(10) Pub. No.: US 2024/0044861 A1
(43) Pub. Date: Feb. 8, 2024

(54) FOOD FRESHNESS DETECTING DEVICE AND METHODS FOR USING THE SAME

(71) Applicant: UNIVERSITY OF MARYLAND, COLLEGE PARK, College Park, MD (US)

(72) Inventor: Cheng GONG, Potomac, MD (US)

(21) Appl. No.: 18/259,019

(22) PCT Filed: Dec. 22, 2021
(86) PCT No.: PCT/US21/65000

§ 371 Date: Jun. 22, 2023

Related U.S. Application Data
(60) Provisional application No. 63/129,523, filed on Dec. 22, 2020.

(51) Int. Cl. G01N 33/12; G01N 33/00

(52) U.S. Cl. CPC G01N 33/12; G01N 33/0036; G01N 33/0031

(57) ABSTRACT

The present disclosure provides a device for detecting food freshness and methods for using the same. In certain embodiments, the device includes (i) a gas sensor system comprising a plurality of gas sensors that produces a plurality of output signals based on the level of gases detected, released by a test food sample, (ii) a detection system that analyzes the plurality of signals produced by the gas sensor system and produces test results for each output signals, and (iii) a display unit that provides useful information based on the test results. In some embodiments, the plurality of gas sensors may include 2D sensors.

### CROSS-REFERENCE TO RELATED APPLICATIONS

This application claims the priority to U.S. Provisional Application No. 63/129,523, filed Dec. 22, 2020, which is hereby incorporated by reference in its entirety.

### FIELD

The present disclosure relates generally to systems, devices, and methods for detecting food freshness. An example device may include (i) a gas sensor system comprising a plurality of gas sensors that produce a plurality of output signals based on the level of gases detected, as released by a test food sample, (ii) a detection system that analyzes the plurality of signals produced by the gas sensor system and produces test results for each output signal, and (iii) a display unit that provides useful information based on the test results.

### BACKGROUND

In 2010, it was estimated that food loss and waste was estimated to be about 31 percent of the total food supply in the U.S. This is equivalent to 133 billion pounds and $162 billion in waste or loss. The top three groups in terms of share of the total value of food loss are meat (30 percent, including poultry and fish), vegetables (19 percent), and dairy products (17 percent). Per the USDA, 21.7 percent of meat that enters the retail market has been lost as “not eaten” at the consumer level, and 4.6 percent at the retailer level.

The common method for companies to suggest the edibility of meat products is via the “sell by” tags on product packages, which is often inaccurate because it does not fully consider the history of a specific product on sale. For example, freshness depends on a variety of factors including, but not limited to, the amount of time a meat product stayed in transportation, the temperature of the transportation environment, etc. In addition, other factors after the product is purchased by a consumer can affect the freshness including, but not limited to, refrigerator temperature, the treatment that leftover meat is subjected to, etc. Without such detailed information, “sell by” tags can convey very limited information to consumers. Based solely on “sell by” tags, spoiled meat may be eaten or good meat may be discarded.

People can generally smell unpleasant odors when meats become spoiled. Thus, gas detection is a practical way for food freshness monitoring. Spoiled food products, such as meats, usually emit or release ammonia (NH₃), trimethylamine (N(CH₃)₃), and/or hydrogen sulfide (H₂S). Although gas sensors exist in industry for other applications (e.g., for toxic gas detection in nanofabrication clean rooms), these facilities/equipment are relatively bulky, of low sensitivity, and/or expensive, thereby making them impractical for food freshness tests in the consumer domain.

While there are a handful of meat freshness sensors on the market for consumers (e.g., Food Sniffer®), they are relatively expensive and easily malfunction when getting close to the spoiled meats due to the strong chemical reactions between gases and the sensor materials, which are typically metal-oxide nanoparticles.

Accordingly, there is a need for portable and inexpensive systems and devices that do not rely on chemical reactions and can help a user quickly determine the freshness of food products. Such systems and devices will aid in food safety and minimize food waste.

### BRIEF SUMMARY

Some aspects of the disclosure provide systems and devices comprising a network of gas sensors. In some embodiments, gas sensors comprise atomic-thin two-dimensional (2D) materials (e.g., graphene, MoS₂, WS₂, WSe₂, etc.). Gas sensors of the disclosure can quickly detect gaseous molecules emitted from spoiled food products, e.g., meats, without requiring chemical reactions. Still in other embodiments, gas sensors of the disclosure are sensitive, inexpensive, and robust.

One particular aspect of the disclosure provides an electronic device or a food freshness detection device 100 for determining food freshness. The electronic device comprises:
a gas sensor 104 system configured to generate a plurality of output signals based on gaseous compounds released by a test food sample, wherein said gas sensor system comprises a plurality of gas sensors 128 configured to detect a level of a gas mixture emitted by said test food sample, and wherein at least some of the said plurality of gas sensors 128 are 2D sensors;
a detection system 108 that is operatively connected to said gas sensor system 104 and configured to receive and process said plurality of output signals to generate a plurality of test results for said gas mixture; and
a display unit 112 operatively connected to said detection system 108 and configured to display freshness of said test food sample based on said plurality of test results.

In some embodiments, the electronic device 100 further includes a memory unit 116. In some instances, the memory unit 116 is configured to store results of said plurality of test results. Still in other instances, the memory unit 116 can be used for storing threshold values that are used for analyzing the plurality of output signals generated by the gas sensor system 104.

Typically, said plurality of output signals comprise electric conductance, electric resistance, or a combination thereof. In some embodiments, gas sensors of the present disclosure generate output signals based on the transfer of electrons between the gases and the sensors. In one particular embodiment, the generation of said plurality of output signals comprises the transfer of electrons from said gas mixture to/from said plurality of gas sensors 128. Accordingly, in some embodiments of the disclosure, the output signals do not depend on any chemical reactions, which is often used in conventional gas sensors.

Yet in other embodiments, said detection system 108 compares each of said plurality of output signals received from said gas sensor system 104 to a corresponding threshold value. The threshold value can be the signal in the absence of the gas being measured, i.e., at ambient conditions without any food sample, or the initial signal can be the signal generated by a food sample that is known to be fresh. Still alternatively, the threshold value can be the signal generated by the food sample at the time of purchase by a user.

In further embodiments, said electronic device 100 includes or is operatively connected to a machine learning system. The machine learning system (i.e., a deep learning system or an artificial intelligence system) can be used to update the threshold values based on said plurality of test results. In this manner, the threshold values can be updated regularly to provide improved detection and sensitivity.

In some embodiments, said machine learning system is remotely located. Alternatively, the machine learning system can be located within the electronic device thereby providing a self-contained unit.

Yet in other embodiments, the electronic device further comprises a network or cloud access system 124 for accessing said machine learning system. Still in other embodiments, said network or cloud access system 124 is configured to store said plurality of test results.

Another aspect of the disclosure provides an electronic meat freshness indicator for determining meat freshness. The meat freshness indicator comprises:
a gas sensor system 104 comprising a plurality of gas sensors 128, each of which is configured to detect a level of a gas mixture emitted by said meat sample and generate a plurality of output signals, wherein said gas mixture comprises ammonia (NH₃), hydrogen sulfide (H₂S), trimethylamine (N(CH₃)₃), and sulfur dioxide (SO₂), and wherein each of said plurality of output signals is an electric conductance, electric resistance, or a combination thereof;
a detection system 108 that is operatively connected to said gas sensor system 104 and configured to receive and process said plurality of output signals to generate a plurality of test results; and
a display unit 112 operatively connected to said detection system 108 and configured to display an indication of meat freshness based on said plurality of test results.

In some embodiments, the detection system 108 comprises a processor that is operatively connected to said gas sensor system 104 and configured to receive and process said plurality of output signals to generate said plurality of test results. In one particular embodiment, said plurality of test results comprises a result for NH₃, H₂S, N(CH₃)₃, and SO₂ gases.

Yet in other embodiments, said detection system 108 compares each of said plurality of output signals received from said gas sensor system 104 to a corresponding threshold value for NH₃, H₂S, N(CH₃)₃, and SO₂ gases.

Still, in other embodiments, the electronic meat freshness indicator further comprises a machine learning system. The machine learning system is configured to evaluate and update each of said threshold values for NH₃, H₂S, N(CH₃)₃, and SO₂ gases.

In further embodiments, the electronic meat freshness indicator further comprises a network or cloud system 124, wherein said machine learning system is remotely located and accessed via said network or cloud system 124.

Yet still in other embodiments, said plurality of gas sensors 128 comprises WS₂, MoS₂, graphene, and WSe₂. In one particular embodiment, said plurality of gas sensors 128 are 2D sensors.

Still another aspect of the disclosure provides a method for determining meat freshness. The method includes:
measuring a level of ammonia (NH₃), hydrogen sulfide (H₂S), trimethylamine (N(CH₃)₃), and sulfur dioxide (SO₂) gases in a gaseous mixture released by a test meat sample using an electronic device or food freshness detection device as disclosed herein; and
determining freshness of said test meat sample by analyzing a test result for each of NH₃, H₂S, N(CH₃)₃, and SO₂ gases.

In some embodiments, the method further includes the steps of placing said test meat sample under a device shielding extraneous odors and creating a defined headspace over said test meat sample and measuring the level of NH₃, H₂S, N(CH₃)₃, and SO₂ gases in said headspace. Still in other embodiments, said device shielding extraneous odors and creating a defined headspace comprises a container with a lid, and one of a cover or funnel tip-cover to isolate said plurality of gas sensors from environment gases.

### BRIEF DESCRIPTION OF THE DRAWINGS

FIG. 1 is a schematic diagram of one particular embodiment of the disclosure having a gas sensor, detection system, and digital output with other optional components.

FIG. 2 is a schematic illustration of one particular embodiment of the gas sensor system of the disclosure comprising graphene, WS₂, WSe₂, and MoS₂ as gas sensors.

FIG. 3. shows electron affinity of various sensors and reduction potential of various gases along with a schematic illustration of one embodiment of multiplex data acquisition enabling an exponentially increased selectivity among 2N gases, where N is the number of different gases.

FIG. 4 is a flow diagram illustrating one particular method, according to examples of the present disclosure.

### DETAILED DESCRIPTION

The present disclosure will be described with regard to the accompanying drawings, which assist in illustrating various features of a system and a device for detecting food freshness. In this regard, the subject matter of the present disclosure generally relates to a gas sensor system for detecting meat freshness. For the sake of clarity and brevity, the subject matter of the present disclosure will now be described in reference to detecting meat freshness. However, it should be appreciated that the scope of the disclosure is not limited to merely detecting meat freshness. In fact, by utilizing different gas sensors, methods and devices described in the present disclosure can be used generally to detect the freshness of any type of food such as fruits, vegetables, bread, dairy products, etc. Discussion on detecting meat freshness is provided solely for the purpose of illustrating the practice of the disclosure and does not constitute limitations on the scope thereof.

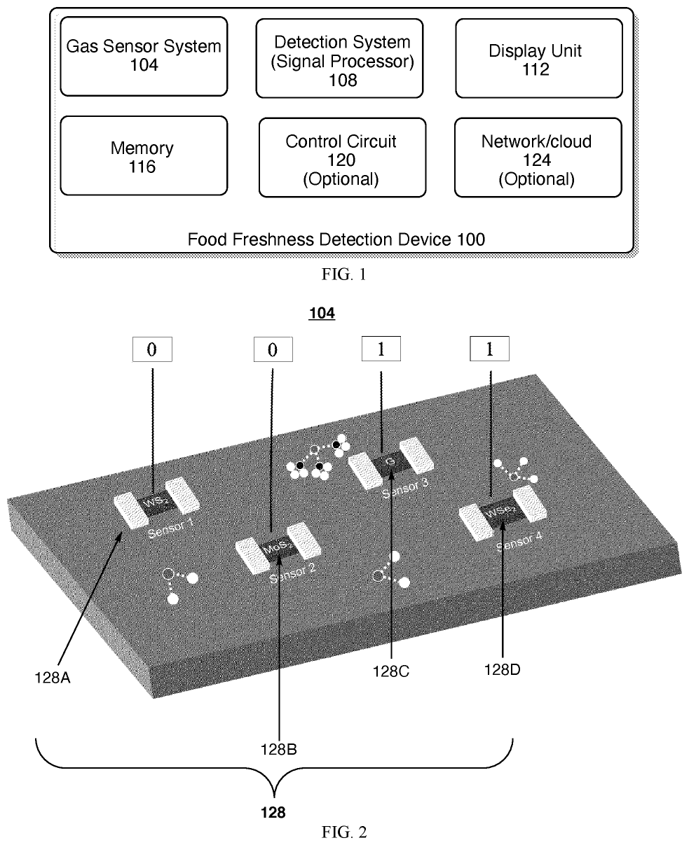

One particular embodiment of a food freshness detection device 100 is schematically illustrated in FIG. 1. As can be seen, in FIG. 1, the food freshness detection device includes a gas sensor system 104, a detection system 108, and a display unit 112. As shown in FIG. 2, the gas sensor system includes a plurality of gas sensors 128. Throughout this disclosure, elements with same numeric value indicate the same elements, for example, elements labeled 128 means a plurality of gas sensors and labels 128A-128D refer to a particular type of individual gas sensor. The plurality of gas sensors 128 are configured to detect a level of a gas mixture released by a test food sample. The particular gas mixture depends on the type of food sample tested. For example, spoiled meats emit a variety of gases including, but not limited to, ammonia, amines, hydrogen sulfide, dimethyl sulfide, trimethylamine, and cadaverine, etc.; and spoiled milk products emit gases such as, but not limited to, ammonia, amines, methane, etc. For meat samples, one can analyze ammonia, sulfur dioxide, trimethylamine, sulfur dioxide, as well as other volatile organics, such as volatile acids (e.g., formic acid, acetic acid, butyric acid, propionic acid, caproic acid, valerian acid, lactic acid, pyruvic acid, etc.), aldehydes, ketones, etc. Accordingly, the type of sensors used in the plurality of gas sensors 128 may depend on the type of food product whose freshness is to be determined. In addition, the type of sensors present in the plurality of gas sensors 128 depends on the configuration of the food freshness detection device 100. For example, if the food freshness detection device 100 is configured only for determining meat freshness, the food freshness detection device 100 can have graphene (128C), MoS₂ (128B), WS₂ (128A) and WSe₂ (128D) as the plurality of gas sensors 128. Alternatively, if the food freshness detection device 100 is configured to detect freshness of a wide variety of food products, it can have an optional control circuit 120 that allows selection of food product whose freshness is to be determined. Selection by this control circuit 120 also selects a particular type of plurality of gas sensors 128 that are present. In this case, other gas sensors in addition to 128A-128D may also be present in the food freshness detection device 100.

In some embodiments, each gas sensor comprises a two-dimensional (2D) material. As used herein, the term “2D material” refers to single-layer materials or solids consisting of a single layer of atoms or crystals. Still in other embodiments, the gas sensor system comprises a hybrid nanosensor network based on various 2D materials for the detection of gaseous molecules emitted (i.e., released) from spoiled meats. It should be appreciated that, unlike conventional gas detection sensors, gas sensors of the disclosure do not rely on a chemical reaction. Gas sensors of the disclosure are highly sensitive and can detect a particular gas at a concentration level of at least about 1 ppm (parts per million), typically at a concentration level of at least about 500 ppb (parts per billion), often at a concentration level of at least about 100 ppb, more often at a concentration level of at least about 50 ppb, still more often at a concentration level of at least about 10 ppb, and most often at a concentration level of at least about 1 ppb.

Without being bound by any theory, it is believed that in some embodiments, the gas sensors of the disclosure rely on gas molecules' physical adsorption on 2D materials, and not on chemical reactions. As used herein, the term “chemical reaction” refers to a process in which one or more substances are converted to one or more different substances. More specifically, the term “chemical reaction” refers to a process where one or more bonds in a molecule is broken and/or formed. It is believed that in gas sensors of the present disclosure, as gas molecules approach 2D materials (i.e., gas sensors), transfer of electron(s) occurs between gas molecules and 2D materials. When 2D materials receive electrons from gas molecules, 2D sensors become electrically more conductive, i.e., electrical resistance decreases compared to 2D sensors in a natural or unmodified state. In contrast, when 2D materials lose or donate electrons to gas molecules, 2D sensors become electrically more resistant, i.e., electrical resistance increases compared to 2D sensors in a natural or unmodified state. Given that 2D materials are of a single crystal layer thickness and easily accessible by gas molecules, the detection sensitivity of gas sensors of the disclosure can be as low as about 100 ppb (particle per billion), typically about 50 ppb, often about 10 ppb, and most often about 1 ppb. This level of selectivity is at least about three orders of magnitude more sensitive than other conventional gas sensors.

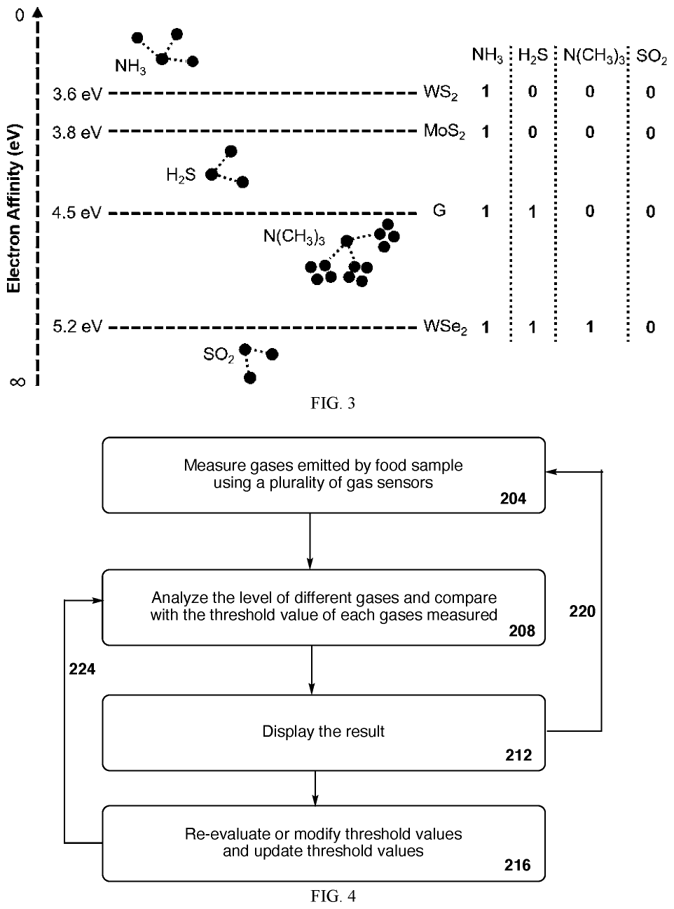

While electrical nanosensors are highly sensitive, they have poor selectivity. Because each individual gas sensor of the disclosure is based on either increased or decreased electrical resistance, it is difficult to distinguish different gases based merely on changes in electrical resistance of a single gas sensor of the disclosure. For example, as can be seen in FIG. 3, when WS₂ sensor (128A) is used, electrons in NH₃ are transferred to WS₂ because WS₂ has a higher electron affinity than NH₃. This transfer of electrons from ammonia to WS₂ results in a decreased electrical resistance in WS₂ sensor (128A). Such a decrease in electrical resistance can be used to detect the level of ammonia. However, other gases, such as H₂S, N(CH₃)₃ and SO₂ have a higher electron affinity compared to WS₂ sensor (128A), therefore electrons are transferred from WS₂ sensor (128A) to one of these gases, thereby causing an increase in electrical resistance in WS₂ sensor (128A). Accordingly, WS₂ sensor (128A) is unable to distinguish H₂S, N(CH₃)₃ and SO₂ gases.

To avoid lack of sensitivity and/or selectivity of gases, in some embodiments, the gas sensor system of the disclosure comprises a hybrid sensor network or a plurality of gas sensors 128. In one particular embodiment, the sensor network includes WS₂, MoS₂, graphene, and WSe₂ sensors ((128A-128D, respectively) for the multiplex data acquisition. Other gas sensors can also be used as a replacement for one of the sensors listed above, or as additional sensor(s) to provide further enhanced selectivity and/or sensitivity. Other suitable gas sensors include, but are not limited to, metal-oxide-semiconductor (MOS) nanoparticles/nanostructures sensors, which are well known to one skilled in the art, as well as electrochemical cells, nanotubes, metal-organic-frameworks.

Use of a network of sensors results in a highly selective and/or sensitive food freshness detection system. For example, the network of sensors illustrated in FIG. 2 will output [1111] for NH₃, [0011] for H₂S, [0001] for N(CH₃)₃, and [0000] for SO₂. Additional advantages of using a network of sensors include, but are not limited to, high selectivity to discern multiple gases, and (2) applicable to different food products, considering different food products emit different gas molecules. This allows consumers to use a single gas sensor system of the disclosure to determine the freshness of any perishable foods including, but not limited to, meats, dairy products, fruits, vegetables, breads, other baked goods, etc.

As discussed above, the plurality of gas sensors 128A-128D are configured to generate a plurality of output signals based on gaseous compounds released by a test food sample. The detection system 108 is operatively connected to the gas sensor system 104. The. detection system 108 is configured to receive and process the plurality of output signals from the plurality of gas sensors 128A-128D to generate a plurality of test results. In some embodiments, the detection system 108 compares each of the corresponding output signals to a corresponding threshold value to produce individual gas sensor test results. The detection system 108 can include a central processing unit (CPU) or a network of CPUs to analyze the signals generated from each of the plurality of gas sensors and evaluate the results based on the type of food being analyzed. Accordingly, in some embodiments, the food freshness detection system 100 can also include a food selector (not shown). The food selector allows analysis of different types of food products for freshness analysis based on particular gases or odors released by the food product. The detection system 108 can also include optional memory 116 unit that can store results as well as detection system parameters for analyzing different types of food products. In some embodiments, the disclosed system and method allow a portable device to easily adapt to a large number of different combinations of sensors.

As can be seen in FIG. 1, food freshness detection device 100 can optionally include a control circuit 120. In some embodiments, the control circuit 120 is used to select the type of food sample to be tested. This selection allows the control circuit 120 to activate appropriate gas sensors 128 and also allows the detection system 108 to utilize appropriate threshold values to produce the test results.

The display unit 112 is operatively connected to the detection system 108 and configured to display the freshness of the test food sample based on the plurality of test results determined by the detection system 108. The display unit 112 can be programmed to output or display different types of freshness information, such as freshness scale (e.g., from 1 to 10), freshness rating (e.g., “good”, “ok”, “bad”, etc.), or other means of conveying the level of freshness of the tested food sample. The type of information displayed by the display unit 112 can be controlled by the control circuit 120.

In some embodiments, the food freshness detection device 100 can optionally include network or cloud system 124. The network or cloud system 124 can be used to allow the food freshness detection device 100 to store and/or retrieve various information, such as threshold values, test results, date, time, food sample tested, etc.

Still, in other embodiments, the food freshness detection device 100 includes a deep learning system, i.e., a machine learning system or an artificial intelligence (AI) system. Such an AI system is well known to one skilled in the art. AI system can be a separate system that can communicate with the food freshness detection system 100 via a network or cloud system 124 which can include wireless communication technologies known to one skilled in the art including, but not limited to, near field communication (NFC) system, BlueTooth®, Wi-Fi, cellular communication system, etc. Alternatively, the AI system can be built into the food freshness detection device 100 such that the entire unit is a self-contained or “free-standing” device.

Because each food product typically releases a wide variety of gases, the AI system allows the device to continually modify and “learn” as more food products are analyzed. In this manner, the sensitivity and/or selectivity of the food freshness detection device 100 can be increased with continued use. This is particularly useful when two or more gases are diametrically opposed in relative electron affinity compared to a gas sensor. As an illustration, when a gas sensor is used to analyze meat freshness, the gas sensor may analyze ammonia, trimethylamine, sulfur dioxide, and hydrogen sulfide. Each concentration of these gases will have a different effect on WS₂ 2D sensor (128A) as discussed above. Depending on the concentration of each of these gases, the change in electric conductivity of WS₂ sensor (128A) can vary slightly. By analyzing many meat samples, the food freshness detection device 100 can distinguish whether the meat is fresh enough for consumption or is spoiled and should not be consumed. In addition, the mixture of these gases will also affect the electric conductance or resistance of other sensors such as graphene, WSe₂ and MoS₂. Using AI, the food freshness detection device can combine the results of all of these sensors to arrive at the most accurate determination of freshness of the meat.

As discussed above, the food freshness detection device 100 can also include control circuit 120. The control circuit 120 can be used for a variety of purposes including, but not limited to, controlling the amount of time the plurality of gas sensors 128 is exposed to gases released by the food product, selecting which gas sensors to activate depending on the food product, selecting the format of the information displayed by the display unit 112, storing the information (e.g., date and time, results, type of food, etc.), as well as other useful information that can be selected by the user.

In certain embodiments, the display unit 112 converts the electronic signal to a user-readable output. The output generated by the digital output of display unit 112 can be as simple as “safe” or “not safe”, or it can be as complex as providing a level of each of one or more gases detected. Such output information can also be configured by the user. In this manner, a wide variety of information can be provided. Display unit 112 can be implemented using one or more computers, one or more servers, one or more databases, one or more cloud computing configurations, and one or more communication networks.

The food freshness detector device 100 (i.e., a gas sensor system) is based on a value of a plurality of electric conductance/resistance signals that are determined using a plurality of gas sensors 128, in particular comprising 2D gas sensors. In some embodiments, the electric conductance is compared to a threshold value in concentrations of gaseous compounds measured from tested food samples to threshold levels to determine food freshness. In one particular embodiment, each of the plurality of gas sensors 128 have a different reduction potential such that an electrical conductance of each of the plurality of gas sensors 128 changes depending on the level of ammonia (NH₃), hydrogen sulfide (H₂S), trimethylamine (N(CH₃)₃), and sulfur dioxide (SO₂).

One particular method of determining food freshness is illustrated in flow diagram shown in FIG. 4. In this method, a plurality of gases emitted or released by a food sample is measured using a plurality of gas sensors (204). The level of released gases is analyzed and compared with the threshold value of each of the measured gases (208). The threshold value can be an initial value of the sensor, i.e., in the absence of the gas emitted or released by the food sample or ambient air. Alternatively, the threshold value can be set by measuring levels of gases of the food sample that is known to be fresh. The results of compared values are then displayed (212) to inform freshness of the food sample. The measurement of food freshness can be repeated (200) or the threshold value is updated (216) based on the result prior to measuring the freshness of another food sample (224).

Additional objects, advantages, and novel features of examples described in the present disclosure will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

Fabrication of devices and scalability for mass production. The variety of 2D materials in the present disclosure are fabricated by chemical vapor deposition (CVD) in 4-inch wafer scale or even larger-size. The different 2D materials are transferred onto the same silicon wafer. Ultraviolet lithography is applied to fabricate the sensor array.

Testing results. Sensitivity tests showed the sensors can detect <1 ppb gases such as NO₂, NH₃, and H₂S in a testing vacuum chamber.

Food test results. A plurality of gas sensors 128 were used to detect gases from spoiled beef, chicken and shrimp. Clear and unambiguous electrical resistance changes were observed compared to fresh meats that did not cause a significant electrical resistance change. The spoiled meat test was also able to distinguish the type of spoiled meat (e.g., beef, chicken or shrimp) based on the different electrical resistance changes (e.g., increase or decrease) and different amplitudes of change caused by each of the different meats.

The foregoing discussion of the subject matter of the present disclosure has been presented for purposes of illustration and description. The foregoing is not intended to limit the the scope of the present disclosure to the form or forms disclosed herein. Although the description of the subject matter of the present disclosure includes the description of one or more embodiments and certain variations and modifications, other variations and modifications are within the scope of the present disclosure, e.g., as may be within the skill and knowledge of those in the art, after understanding the present disclosure. It is intended to obtain rights, which include alternative embodiments to the extent permitted, including alternate, interchangeable, and/or equivalent structures, functions, ranges or steps to those claimed, whether or not such alternate, interchangeable, and/or equivalent structures, functions, ranges or steps are disclosed herein, and without intending to publicly dedicate any patentable subject matter. All references cited herein are incorporated by reference in their entirety.

1. A food freshness detection device for determining food freshness, said food freshness detection device comprising:
a gas sensor system configured to generate a plurality of output signals based on gaseous compounds released by a test food sample, wherein said gas sensor system comprises a plurality of gas sensors configured to detect a level of a gas mixture emitted by said test food sample, and wherein at least some of said plurality of gas sensors are 2D sensors;
a detection system that is operatively connected to said gas sensor system and configured to receive and process said plurality of output signals to generate a plurality of test results for said gas mixture; and
a display unit operatively connected to said detection system and configured to display freshness of said test food sample based on said plurality of test results.

2. The food freshness detection device of claim 1, further comprising a memory unit.

3. The food freshness detection device of claim 2, wherein said memory unit is configured to store results of said plurality of test results.

4. The food freshness detection device of claim 1, wherein said plurality of output signals comprise electric conductance, electric resistance, or a combination thereof.

5. The food freshness detection device of claim 1, wherein generation of said plurality of output signals comprise transfer of electrons from said gas mixture to or from said plurality of gas sensors.

6. The food freshness detection device of claim 1, wherein said detection system compares each of said plurality of output signals received from said gas sensor system to a corresponding threshold value.

7. The food freshness detection device of claim 6, wherein said food freshness detection device is operatively connected to a machine learning system, whereby said threshold value is updated based on said plurality of test results.

8. The food freshness detection device of claim, 7 further comprising a network or cloud access system for accessing said machine learning system.

9. The food freshness detection device of claim 8, wherein said network or cloud access system is configured to store said plurality of test results.

10. The food freshness detection device of claim 7, wherein said machine learning system is located within said food freshness detection device.

11. An electronic meat freshness indicator for determining meat freshness, said electronic meat freshness indicator comprising:
a gas sensor system comprising a plurality of gas sensors, each of which is configured to detect a level of a gas mixture emitted by said meat sample and generate a plurality of output signals, wherein said gas mixture comprises ammonia (NH₃), hydrogen sulfide (H₂S), trimethylamine (N(CH₃)₃), and sulfur dioxide (SO₂), and wherein each of said plurality of output signals is an electric conductance, electric resistance, or a combination thereof;
a detection system that is operatively connected to said gas sensor system and configured to receive and process said plurality of output signals to generate a plurality of test results; and
a display unit operatively connected to said detection system and configured to display an indication of meat freshness based on said plurality of test results.

12. The electronic meat freshness indicator of claim 11, wherein said detection system comprises a processor that is operatively connected to said gas sensor system and configured to receive and process said plurality of output signals to generate said plurality of test results.

13. The electronic meat freshness indicator of claim 11, wherein said plurality of test results comprises a result for NH₃, H₂S, N(CH₃)₃, and SO₂ gases.

14. The electronic meat freshness indicator of claim 11, wherein said detection system compares each of said plurality of output signals received from said gas sensor system to a corresponding threshold value for NH₃, H₂S, N(CH₃)₃, and SO₂ gases.

15. The electronic meat freshness indicator of claim 14, further comprising a machine learning system, wherein said machine learning system is configured to evaluate and update each of said threshold values for NH₃, H₂S, N(CH₃)₃, and SO₂ gases.

16. The electronic meat freshness indicator of claim 15, further comprising a network system, wherein said machine learning system is remotely located and accessed via said network system.

17. The electronic meat freshness indicator of claim 11, wherein said plurality of gas sensors comprises WS₂, MoS₂, graphene, and WSe₂.

18. The electronic meat freshness indicator of claim 17, wherein said plurality of gas sensors are 2D sensors.

19. A method for determining meat freshness, said method comprising:
measuring a level of ammonia (NH₃), hydrogen sulfide (H₂S), trimethylamine (N(CH₃)₃), and sulfur dioxide (SO₂) gases in a gaseous mixture released by a test meat sample using a food freshness detection device of claim 1; and
determining freshness of said test meat sample by analyzing a test result for each of NH₃, H₂S, N(CH₃)₃, and SO₂ gases.

20. The method of claim 19, further comprising the steps of placing said test meat sample under a device shielding extraneous odors and creating a defined headspace over said test meat sample and measuring the level of NH₃, H₂S, N(CH₃)₃, and SO₂ gases in said headspace.

21. The method of claim 20, wherein said device shielding extraneous odors and creating the defined headspace comprises a container with a lid, and one of a cover or funnel tip-cover to isolate said plurality of gas sensors from environment gases.
